(19) United States
(12) Patent Application Publication
(10) Pub. No.: US 2013/0267790 A1
(43) Pub. Date: Oct. 10, 2013

(54) ELECTRONIC ADHESIVE BANDAGE DEVICE WITH AT LEAST ONE ENERGY SOURCE

(71) Applicant: ROBERT BOSCH GMBH, Stuttgart (DE)

(72) Inventors: Andre Pfuetzner, Heilsbronn (DE); Henning Hayn, Stuttgart (DE)

(73) Assignee: Robert Bosch GmbH, Stuttgart (DE)

(21) Appl. No.: 13/859,591

(22) Filed: Apr. 9, 2013

(30) Foreign Application Priority Data
Apr. 10, 2012 (DE) 10 2012 205 745.5

(51) Int. Cl. A61B 5/00

(52) U.S. Cl. CPC A61B 5/6832
USPC 600/300

(57) ABSTRACT

An electrical adhesive bandage device for application on the skin includes a first section and at least one second section. The first section has a functional apparatus which has electronics, and the at least one second section has at least one energy source which is connected to the functional apparatus and the electronics thereof. The first section and the last least one second section are arranged next to one another and configured to be separable from one another by at least one separation edge.

This application claims priority under 35 U.S.C. §119 to patent application no. DE 10 2012 205 745.5, filed on Apr. 10, 2012 in Germany, the disclosure of which is incorporated herein by reference in its entirety.

The disclosure relates to an electronic adhesive bandage device with at least one energy source, for application to skin and more particularly for application to human or animal skin.

### BACKGROUND

Electronic functional adhesive bandages are adhesive bandages with electronic circuits, which are used for e.g. monitoring human or else animal vital parameters. The circuits consist of electronic components such as e.g. sensors, actuators, microprocessors or ASICs for signal processing and for controlling batteries, display elements and operating elements of the adhesive bandage.

Electronic adhesive bandages are currently being developed for a multiplicity of applications. Known applications include EKG (electrocardiogram) measurements, EMG (electromyography) measurements or measurements of the blood oxygen saturation. By way of example, further applications are adhesive bandages for measuring the daily calorie intake or an adhesive bandage for pain therapy. Moreover, there are electronic adhesive bandages for acoustic detection of sleep apnea.

All of these adhesive bandages with electrical or electronic components require an energy supply and hence an energy source, which, for example, can be embodied as a battery in the form of a button cell. Here, the battery is either inseparably connected to the electronics and integrated into the adhesive bandage or configured to be replaceable. In the case of such batteries integrated into the adhesive bandage, the whole adhesive bandage must at a later stage be disposed of in accordance with the battery ordinance.

Moreover, WO 1996030077 A1 has disclosed a transdermal active ingredient releasing device, which has a first upper and a second lower housing. The first housing contains electronic components and a battery, and the second housing contains interacting electrodes and reservoirs. The first and the second housing can be separated by a strap. Moreover, provision is made for a connecting housing, which comprises means for contacting the battery in the connecting housing and a curved slit for simplifying a manual removal of the battery from the connecting housing.

### SUMMARY

The disclosure provides an electronic adhesive bandage device according to the description below.

The subjects of the description relate to preferred developments.

The electronic adhesive bandage device according to the disclosure allows a very simple separation of an energy source from the remaining part of the adhesive bandage device, which comprises a functional apparatus with electronics. As a result, a simplified disposal of the energy source is possible since the latter can reliably be separated from the adhesive bandage device. In particular, this renders possible a disposal according to the waste ordinance.

The concept underlying the present disclosure consists of subdividing the adhesive bandage device into at least two sections which are situated next to one another, wherein one section comprises the functional apparatus with the electronics and the other section comprises the at least one energy source, and wherein the two sections are separable from one another by means of a separation edge. Here, the separation edge is firstly simple and cost-effective in terms of its production. Moreover, the tearing open of the adhesive bandage device can be controlled by the separation edge and made easier for a user since he has to apply less force and the separation edge guides the separation in such a way that the adhesive bandage device is separated at the connection of the two sections with the functional apparatus and the energy source, respectively. This prevents a separation at a different position.

In one embodiment, the energy source can be connected to the functional apparatus by means of at least one line, wherein the line is, for example, a printed conductor track, a conductor wire and/or a conductor cable.

In a preferred embodiment, the line has a connection to the functional apparatus and a connection to the energy source, wherein the connection of the line to the functional apparatus is attached or affixed more securely than the connection of the line to the energy source. This can additionally simplify a separation of the line from the energy source when the section with the energy source is separated from the section with the functional apparatus because the connection of the line to the energy source can be detached more easily.

In a further embodiment, the connection of the line to the energy source is attached in such a way that, when the second section is torn off the first section, it can be torn off the energy source and the connection of the line to the functional apparatus is attached in such a way that, when the second section is torn off the first section, it remains connected to the electronics. As a result, it is additionally possible to ensure that the line remains on the functional apparatus when separating the section with the energy source and is not separated together with the energy source.

The separation edge can optionally be provided with an additional notch at at least one end. The notch eases or simplifies the tearing apart of the adhesive bandage device at the separation edge. By way of example, the notch can have an angled, round, slit-shaped, drop-shaped, u-shaped and/or semicircular form.

In a preferred embodiment, the separation edge can be perforated, configured as a fold and/or configured to be dissolvable. The adhesive bandage device can be weakened at this point in a targeted fashion by means of perforations and/or the fold and thereby simplify a tearing apart of the adhesive bandage device by a user or wearer. Configuring the separation edge in such a way that the latter dissolves or dissolves by itself within a defined or predetermined period of time is in turn advantageous in that a user or wearer of the adhesive bandage device can easily completely tear off the section with the energy source, particularly if the adhesive bandage device is attached to a body site which is difficult for him or her to see or hard to reach. Here, in one embodiment, the adhesive bandage device can be configured to be dissolved by a chemical and/or biological reaction. In particular, the separation edge can be configured to be dissolvable within a predetermined period of time as a result of the contact with air, water and/or as a result of the body temperature of the adhesive bandage wearer.

In one embodiment, at least one of the connections to the functional apparatus and the energy source, respectively, is a soldering point and/or a clamping contact. Such soldering points and clamping contacts are easy and cost-effective in terms of their production and moreover render it possible to configure the connection of the line to the functional apparatus in such a way that the line cannot be separated from the functional apparatus, or cannot be separated from the functional apparatus without additional aids such as e.g. scissors, when the section with the energy source is torn off the section with the functional apparatus. Secondly, such soldering points and clamping contacts in turn render it possible to configure the connection of the line to the energy source in such a way that the line can easily or only by applying manual force be separated from the energy source when the section with the energy source is separated from the section with the functional apparatus.

In one embodiment, the adhesive bandage device comprises at least one additional third section with at least one energy source, wherein the first section with the functional apparatus is, in particular, arranged between the second and the third section and the second and the third section can be separated from the first section with the functional apparatus by means of at least one separation edge in each case.

In a further embodiment, the adhesive bandage device has an angled shape, more particularly a quadrilateral or triangular shape, a round shape, more particularly an elliptical shape or a circular shape and/or a bonbon shape.

### BRIEF DESCRIPTION OF THE DRAWINGS

Further features and advantages of the present disclosure will be explained below on the basis of the figures. In detail:

FIG. 1 shows a schematic illustration of an embodiment of an electronic adhesive bandage device according to the disclosure with two energy sources;

FIG. 2 shows the electronic adhesive bandage device in accordance with FIG. 1, wherein the energy sources are partly or completely separated;

FIGS. 3A-3C show further exemplary embodiments of the electronic adhesive bandage device according to the disclosure; and

FIGS. 4A-4E show various exemplary embodiments of geometries of notches for separating the respective energy source from the adhesive bandage device.

### DETAILED DESCRIPTION

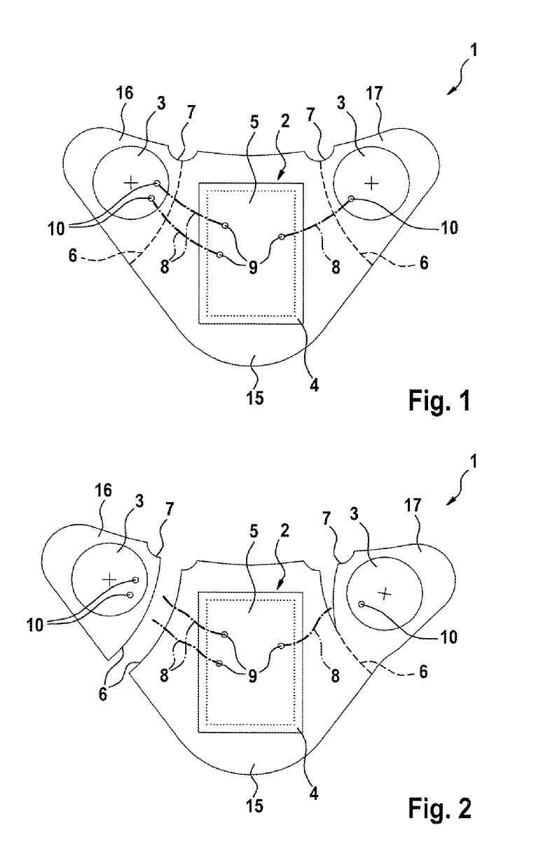

FIG. 1 shows a schematic illustration of an embodiment of an electronic adhesive bandage device 1 according to the disclosure, having at least one functional apparatus 2 and two separable energy sources 3. The adhesive bandage device in this case has a first section 15 with the functional apparatus 2 and a second and third section 16, 17 with respectively one energy source 3, wherein all sections 15, 16, 17 are arranged next to one another and connected to one another in a separable fashion by means of a separation edge 6.

In the exemplary embodiment shown in FIG. 1, the adhesive bandage device 1 has a triangular shape, wherein, for example, the first section with the at least one functional apparatus 2 is arranged between the second and third section with the two energy sources 3.

However, the disclosure is not restricted to this specific embodiment. In principle, the adhesive bandage device 1 can have any shape and can have at least one first section with the functional apparatus and a second section with at least one energy source, wherein the sections are arranged next to one another and the section with the energy source is connected to the section with the functional apparatus in a separable fashion by means of a separation edge. This applies to all embodiments of the disclosure and, in particular, to the exemplary embodiments of the disclosure illustrated in FIG. 1 and the subsequent FIGS. 2 to 4.

By way of example, a button cell can be used as energy source 3, as shown in the exemplary embodiment in FIG. 1. However, in addition to a button cell, it is also possible to use any other energy source which is suitable for being connected to the adhesive bandage device and the respective functional apparatus thereof and which provides a sufficient amount of energy for the respectively associated functional apparatus.

In the exemplary embodiment shown in FIG. 1, the functional apparatus 2 of the adhesive bandage device 1 has a carrier apparatus 4, e.g. a printed circuit board, on which electronics are arranged, as indicated by a dotted line, for carrying out a respective function or functions of the adhesive bandage device 1. By way of example, such a function comprises the measurement and optionally the display of a vital parameter of a user of the adhesive bandage device 1, such as, for example, the blood pressure, the blood oxygen saturation, the respiratory rate or the calorie intake etc. The two energy sources 3 are, on the adhesive bandage device 1, provided separately from the functional apparatus 2. In addition to carrying out the function, the functional apparatus 2 and the electronics thereof can moreover comprise further apparatuses such as, for example, a display apparatus, an evaluation apparatus, an operating apparatus etc., to give but a few examples without being restricted to these.

As shown in the exemplary embodiment in FIG. 1, the sections 16 and 17 of the two energy sources 3 are connected to the section 17 of the functional apparatus 2 in a separable fashion. For the purposes of separating the energy source 3 from the functional apparatus 2, the sections 16 and 17, respectively, of the energy source 3 and the section 15 of the functional apparatus 2 are connected to one another such that it is possible to tear them apart by means of a separation edge 6 in order to tear the energy source 3 off the adhesive bandage device 1 and the functional apparatus 2 thereof along the separation edge 6.

Here, the separation edge 6 can, at the beginning, optionally be provided with an additional notch 7 for the purposes of further simplification of the separation of the energy source 3 along the separation edge 6. By way of example, in the exemplary embodiment, as shown in FIG. 1, the notch 7 is configured as an arc-shaped or semicircular recess or indent in the adhesive bandage device 1. The separation edge 6 can in turn be formed by perforations in the adhesive bandage device 1 and/or by a fold or by pressing a flute into the adhesive bandage device 1.

The respective energy source 3 is connected to the functional apparatus 2 by means of at least one line 8, for example a cable or a line wire etc. The respective line 8 and the connections 9 and 10 thereof are indicated in the exemplary embodiment by a dash-dotted line in FIG. 1. Here, the respective connection 9 for the line 8 to the functional apparatus 2 or the electronics, for example a soldering point or a clamping contact, is configured in such a way that the line 8 cannot be separated or can only be separated with great difficulties by the tensile and/or shearing forces caused during the tearing. In turn, the respective connection 10 of the line 8 to the energy source 3, for example a soldering point or a clamp, is embodied in such a way that it can easily be detached by the force which the user of the adhesive bandage device 1 can apply manually without the use of additional aids, such as e.g. scissors, a knife etc.

The separated energy source 3 can subsequently be disposed independently of the remaining adhesive bandage device 1 and the functional apparatus 2 thereof; in particular, it can be disposed pursuant to the battery ordinance. As a result of the provision of the separation edge 6 on the adhesive bandage device 1, it is possible, firstly, to achieve a separation, which can easily be carried out manually by the user and only requires little force input by him, of the respective energy source 3 from the remainder of the adhesive bandage device 2. Secondly, such a separation edge 6 can be realized in a simple and cost-effective manner. The same applies to the optional additional notch 7 on the adhesive bandage device at the start of the separation line.

FIG. 2 shows the electronic adhesive bandage device 1 in accordance with FIG. 1, wherein the second and third sections 16, 17 with the energy sources 3 are partly or completely separated from the first section 15 of the functional apparatus 2. Here, the second section 16 with the energy source 3 is, along the notch 7 and the separation edge 6 connected to the notch 7, completely torn off the first section 15 with the functional apparatus 2. In the process, the two lines 8, by means of which the energy source 3 was connected to the functional apparatus 2, are also separated from the energy source 3, while the lines 8 with the connections thereof are still connected to the functional apparatus 2. As described above, the respective connection for the line 8 to the functional apparatus 2 or the electronics is embodied in such a way that the line 8 cannot be separated, or can only be separated with great difficulties, by the forces caused during the tearing. By contrast, the respective connection of the line 8 to the energy source 3 is embodied in such a way that it can easily be separated manually from the energy source 3, i.e. by the manual strength of the user without the latter having to use additional aids, such as, for example, scissors, a knife or pliers etc.

In the exemplary embodiment shown in FIG. 2, the third section 17 with the energy source 3 is partly separated from the first section 15 with the functional apparatus 2. To this end, part of the separation edge 6 is torn apart in order to separate the part of the adhesive bandage device 1 with the second energy source 3 from the part of the adhesive bandage device 1 with the functional part 2. Here, the energy source 3 is also separated from the respective line 8, by means of which it is connected to the functional apparatus 2, since the connection of the line 8 to the energy source 3 is configured in such a way that it can be separated as easily as possible, i.e. by means of the manual strength of the user only. The perforated and/or folded separation edge 6 is advantageous in that the energy source 3 can easily be separated in a defined manner along this separation or tear edge 6. Moreover, the separation edge 6 prevents the energy source from being separated in an uncontrolled manner along a tear line which occurs arbitrarily during tearing. Instead, the separation edge 6 controls or determines the tear line and, as a result, significantly simplifies the process of tearing the energy source 3 off the adhesive bandage device 1.

In addition to a cable or a line wire as a line 8 for connecting the respective energy source 3 to the functional apparatus 2, provision can just as well be made for a printed conductor track (not illustrated) or a plug-in connection (not illustrated) etc.

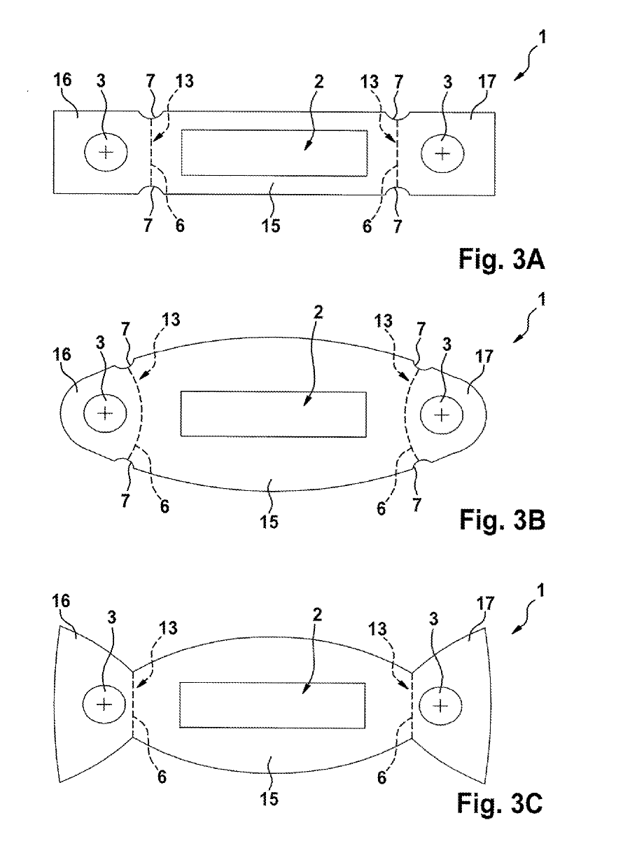

FIGS. 3A to 3C show further exemplary embodiments of the electronic adhesive bandage device 1 according to the disclosure. By way of example, the adhesive bandage devices 1 shown in the exemplary embodiments have a first section with the functional apparatus 2 and a second and third section 16, 17 with respectively one energy source 3, as was already described above with reference to the exemplary embodiment in FIGS. 1 and 2, and so a repetition of the description in this respect is refrained from.

Here, FIG. 3A shows an exemplary embodiment, in which the adhesive bandage device 1 has a quadrilateral, for example rectangular, shape. Here, the two sections 16, 17 with the respective energy source 3 are, for example, arranged at the two outer ends of the rectangular adhesive bandage device 1. The section 15 with the functional apparatus 2 is arranged between the two sections 16 and 17 with the respective energy source 3. The respective energy source 3 is connected to the functional apparatus 2 by means of at least one line. Moreover, the section 15 of the adhesive bandage device 1 is connected to the sections 16 and 17 with the respective energy source 3 in a separable fashion by means of a separation edge 6. In the exemplary embodiment shown in FIG. 3A, the separation edge 6 has a straight-lined profile and is moreover configured for separation of the section 16 or 17 with the energy source 3 by means of perforations 13 in the adhesive bandage device 1. Moreover, an additional notch 7 for further simplification of the separation of the section 16 or 17 of the adhesive bandage device 1 with the energy source 3 is respectively provided at both ends of the separation edge 6. Here, the notch 7 has an arc-shaped or semicircular shape. Instead of a rectangular shape, the adhesive bandage device 1 can also have any other angular shape, for example a square shape, giving but one example of an angular or polygonal shape.

FIG. 3B shows an exemplary embodiment of an adhesive bandage device 1, which has a round and more particularly elliptical shape or kidney shape. Here, the two sections 16 and 17 with the respective energy source 3 are likewise arranged at e.g. the two outer ends of the elliptical adhesive bandage device 1. The section 17 with the functional apparatus 2 is once again arranged between the two sections 16 and 17 with the respective energy sources 3. Furthermore, the respective energy source 3 is connected to the functional apparatus 2 by means of at least one line. The section 16 or 17 of the adhesive bandage device 1 with the energy source 3 is embodied separably from the remaining part of the adhesive bandage device 1 by means of a separation edge 6, which for example has an arc-shaped profile. In the exemplary embodiment shown in FIG. 3B, the separation edge 6 likewise has perforations 13 for tearing apart the adhesive bandage device 1 along the separation edge 6. Here, the separation edge 6 is provided at both ends with an additional notch 7 for easier tearing into the separation edge 6. By way of example, the respective notch 7 is in this case embodied in an arc or semicircular shape.

Furthermore, FIG. 3C illustrates an exemplary embodiment of an adhesive bandage device 1, which has a bonbon shape or a first elliptical or central section with a second and third outer section 16 and 17 in the shape of wings. Here, the two energy sources 3 are arranged in the wing-shaped sections 16 and 17 of the adhesive bandage device 1 and the functional apparatus 2 is arranged in the central elliptical section 15 of the adhesive bandage device 1. The respective energy source 3 is moreover connected to the functional apparatus 2 by means of at least one line. As shown in the exemplary embodiment in FIG. 2, the wing section 16 or 17 of the adhesive bandage device 1 with the respective energy source 3 is configured separably from the remaining part of the adhesive bandage device 1 by means of, for example, a separation edge 6 which extends in a straight line. In the exemplary embodiment shown in FIG. 3C, the separation edge 6 likewise has perforations 13 for tearing open the adhesive bandage device 1 along the separation edge 6, but it does not have an additional notch at one or both ends. Rather, the wing-shaped end sections 16 and 17 themselves act like a notch and allow simple tearing into the separation edge 6 and a separation of the part of the adhesive bandage device 1 with the energy source 3 from the remaining part of the adhesive bandage device 2.

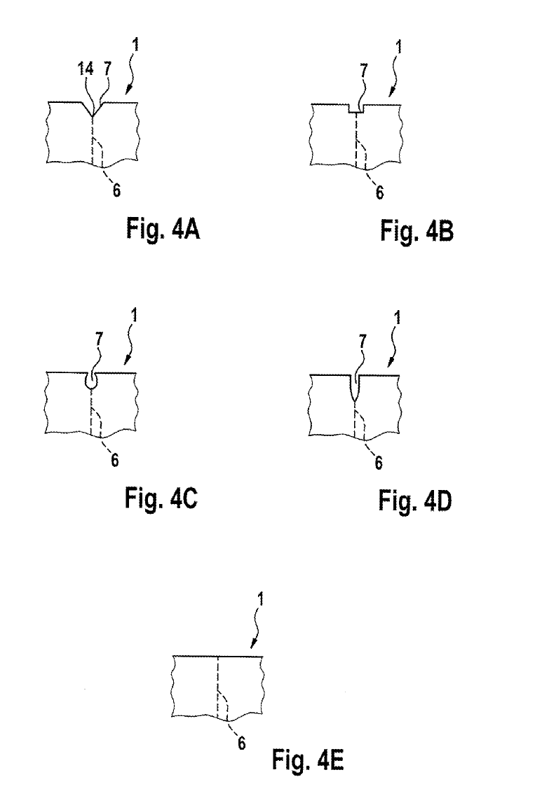

FIGS. 4A to 4E illustrate various exemplary embodiments of geometries of notches 7 for separating the respective energy source from the adhesive bandage device 1.

Here, FIG. 4A shows a section of an adhesive bandage device 1 according to the disclosure, which has a separation edge 6 for separating the part of the adhesive bandage device 1 with the respective energy source from the remaining part of the adhesive bandage device 1. Here, the separation edge 6 has a V-shaped or triangular notch 7 at one end, wherein the tip 14 of the notch 7 preferably points at the separation edge 6. An advantage of the notch 7 is that it additionally makes easier and simplifies the tearing into the separation edge 6. As already described previously, the separation edge 6 is, for example, perforated and/or formed with a fold or a pressed-in flute in the adhesive bandage device 1.

FIG. 4B shows the section of the adhesive bandage device 1 according to the disclosure in accordance with FIG. 4A, wherein, however, the notch 7 in this case has an angular, for example square or rectangular, configuration. The notch 7 makes it simpler to tear open the separation edge 6 and hence to separate the section of the adhesive bandage device 2 with the respective energy source from the remaining adhesive bandage device 1.

A further example of the shape of the notch 7 is shown in FIG. 4C. FIG. 4C shows the section of the adhesive plaster device 2 according to the disclosure in accordance with FIG. 4A, wherein, however, the notch 7 in this case has a round, for example drop-shaped, configuration.

In addition to being drop shaped, the notch 7 can also have a U-shaped configuration, in particular, it can be embodied as an elongate U-shaped notch, as shown in FIG. 4D. FIG. 4D likewise shows the section of the adhesive bandage device 1 according to the disclosure in accordance with FIG. 4A, wherein, however, the notch 7 in this case has a U-shaped configuration, as described above.

FIG. 4E shows the section of the adhesive bandage device 1 according to the disclosure in accordance with FIG. 4A, wherein, however, the separation edge 6 does not have an additional notch at either of its ends.

The separation edge for separating the section of the adhesive bandage device with the respective energy source from the remaining adhesive bandage device, which was described above with reference to the exemplary embodiments in FIGS. 1 to 4, can be perforated, can be embodied with a fold and/or can be embodied to be separable after a certain amount of time. By way of example, the separation edge can be embodied in such a way that it dissolves on its own after a certain amount of time, for example as a result of a chemical and/or biological reaction with the surroundings, for example a reaction with the air and/or water, and thereby allows the energy source to be torn off the adhesive bandage device in a simple manner. This chemical and/or biological reaction can optionally additionally be excitable by the body temperature of the wearer of the adhesive bandage device and the separation edge can thus dissolve on its own accord after a certain amount of time and enable the energy source to be torn off the adhesive bandage device in a simple manner.

Although the present disclosure was completely described above on the basis of preferred exemplary embodiments, it is not restricted thereto, but rather can be modified in various ways.

In the exemplary embodiments described above with reference to FIGS. 1 to 4, and in further exemplary embodiments of the disclosure, the separation edge for separating the section with the at least one energy source from the remaining part of the adhesive bandage device can be perforated, embodied as a fold and/or embodied such that it can dissolve. Here, the separation edge can be configured in such a dissolvable or inherently dissolvable fashion that it preferably dissolves after a predetermined or defined period of time. Here, the adhesive bandage device can be configured to be dissolvable in further embodiments by means of a chemical and/or biological reaction. In particular, the separation edge can be configured to dissolve within a predetermined period of time as a result of the contact with air, water, and/or as a result of the body temperature of the adhesive bandage wearer.

What is claimed is:

1. An electrical adhesive bandage device for application on the skin, comprising:
at least one first section with a functional apparatus which has electronics; and
at least one second section with at least one energy source which is connected to the functional apparatus and the electronics,
wherein the at least one first section and the at least one second section are arranged next to one another and configured to be separable from one another by at least one separation edge.

2. The electrical adhesive bandage device according to claim 1, wherein the at least one energy source is connected to the functional apparatus by at least one line.

3. The electrical adhesive bandage device according to claim 2, wherein the at least one line has a first connection to the functional apparatus and a second connection to the at least one energy source, and the first connection is attached more securely than the second connection.

4. The electrical adhesive bandage device according to claim 3, wherein:
the second connection is attached such that, when the at least one second section is torn off the at least one first section, the second connection is torn off the energy source, and
the first connection is attached such that, when the at least one second section is torn off the at least one first section, the first connection remains connected to the electronics.

5. The electrical adhesive bandage device according to claim 1, wherein the separation edge has an additional notch at at least one end, and the notch has at least one of an angled form, a round form, a slit-shaped form, a drop-shaped form, a u-shaped form, and a semicircular form.

6. The electrical adhesive bandage device according to claim 1, wherein:
the separation edge is at least one of perforated, configured as a fold, and configured to be dissolvable, and
when the separation edge is configured to be dissolvable, the separation edge is configured to be dissolved by at least one of a chemical reaction and a biological reaction.

7. The electrical adhesive bandage device according to claim 2, wherein the line is at least one of a printed conductor track, a conductor wire, and a conductor cable.

8. The electrical adhesive bandage device according to claim 3, wherein at least one of the first connection and the second connection is at least one of a soldering point and a clamping contact.

9. The electrical adhesive bandage device according to claim 1, further comprising:
at least one third section with at least one energy source,
wherein the at least one first section with the functional apparatus is arranged between the at least one second section and the at least one third section, and
wherein the at least one second section and the at least one third section are each separable from the at least one first section with the functional apparatus by at least one separation edge.

10. The electrical adhesive bandage device according to claim 1, wherein the adhesive bandage device has at least one of an angled shape, a round shape, and a bonbon shape.
